(19) United States
(12) Patent Application Publication
(10) Pub. No.: US 2017/0021009 A1
(43) Pub. Date: Jan. 26, 2017

(54) Heat Inactivated Poxvirus Improves Vaccination Results

(71) Applicant: ARIZONA BOARD OF REGENTS ON BEHALF OF ARIZONA STATE UNIVERSITY, Scottsdale, AZ (US)

(72) Inventors: Bertram Jacobs, Tempe, AZ (US); Trung Huynh, Chandler, AZ (US); Negin Blattman, Scottsdale, AZ (US); Keith Dumbell, West Cape (ZA)

(73) Assignee: ARIZONA BOARD OF REGENTS ON BEHALF OF ARIZONA STATE, Scottsdale, AZ (US)

(21) Appl. No.: 15/125,024

(22) PCT Filed: Mar. 10, 2015
(86) PCT No.: PCT/US15/19736

§ 371 Date: Sep. 9, 2016

Related U.S. Application Data
(60) Provisional application No. 61/950,494, filed on Mar. 10, 2014.

(51) Int. Cl. A61K 39/285; C12N 7/00

(52) U.S. Cl. CPC A61K 39/285; C12N 7/00; A61K 2039/5252

(57) ABSTRACT

Compositions including a heat-inactivated poxvirus, such as vaccinia virus, that induces one or more interferons in a vaccine recipient. The compositions may further include one or more live vaccine vectors and non-replicating vaccine components. Methods of vaccine preparation and administration including a heat-inactivated poxvirus, such as vaccinia virus, improve vaccine immunogenicity and safety.

### CROSS-REFERENCE TO RELATED APPLICATIONS

This application claims priority to U.S. Provisional Patent Application No. 61/950,494 filed on Mar. 10, 2014.

### FIELD OF THE INVENTION

This application relates to the field of vaccination against infectious disease and more specifically to the use of heat-inactivated poxvirus in vaccines.

### BACKGROUND OF THE INVENTION

Active immunization involves administration of vaccines containing antigenic molecules (or genes for these molecules) derived from infectious agents. Vaccinated animals react with acquired immune responses and develop prolonged immunity to those agents. When properly used, vaccines are highly effective in controlling infectious diseases.

Vaccines may contain either living or killed organisms or purified antigens from these organisms. Vaccines containing living organisms tend to trigger the best protective responses. Killed organisms or purified antigens may be less immunogenic than living ones. As a result, vaccines that contain killed organisms or purified antigens usually require the use of adjuvants to maximize their effectiveness. Adjuvants may, however, cause local inflammation, and multiple doses or high doses of antigen increase the risks of producing hypersensitivity reactions.

Vaccinia virus was and is still being used as a live-virus vaccine against smallpox virus, which is genetically related to vaccinia. A vaccinia virus infection is very mild and is typically asymptomatic in healthy individuals, but it may cause a mild rash and fever. Moreover, certain complications and/or vaccine adverse effects occasionally arise. The chance of this happening is significantly increased in people who are immunocompromised.

### SUMMARY OF THE INVENTION

This disclosure relates to compositions including a heat-inactivated poxvirus, such as vaccinia virus, that induces one or more interferons, a potent antiviral and immune stimulator. Mixing heat inactivated vaccinia virus with live, virulent vaccinia virus protects animals from lethal infection and still allows induction of a protective immune response.

This disclosure further relates to methods of vaccine preparation and administration including a heat-inactivated poxvirus, such as vaccinia virus.

These and other aspects of the invention will be apparent upon reference to the following detailed description and figure. All references cited throughout are hereby incorporated by reference herein.

### DETAILED DESCRIPTION OF THE INVENTION

Embodiments described herein relate to compositions including heat-inactivated poxvirus, e.g., vaccinia virus, and to methods of preparation and administration of same.

Vaccinia virus contains within its genome several proteins that give the virus resistance to interferons. For example, K3L is a protein with homology to the protein eukaryotic initiation factor 2 (eIF-2alpha). K3L protein inhibits the action of PKR, an activator of interferons. E3L is another protein encoded by Vaccinia. E3L also inhibits PKR activation.

Thus in one novel aspect, it has been discovered that heat-inactivation of a poxvirus allows it to induce interferon, a potent antiviral and immune stimulator, and thereby produce an improved vaccination response in a vaccinated subject.

In another embodiment, heat inactivated poxvirus (e.g., vaccinia virus) is mixed with one or more of a live, virulent virus such that a recipient such as an animal or human is both protected from lethal infection and produces a protective immune response.

In a more specific embodiment, it has been discovered that heat inactivation of vaccinia virus allows it to induce interferon, a potent antiviral and immune stimulator. Moreover, a vaccine composition could be a mixture of heat inactivated vaccinia virus and one or more of live vaccine vectors or nonreplicating vaccine components.

Because killed or inactivated pathogens do not replicate, they typically cannot revert to a more virulent form capable of causing disease. However, inactivated pathogens tend to provide a shorter length of protection than live vaccines, and are more likely to require boosters to create long-term immunity. However, heat-inactivated poxvirus has been discovered to confer better immunogenecity.

The poxvirus may be heat inactivated by one of several known means. For example, the virus may inactivated by dry heat at 95° C. for 2 hours or by moist heat at 60 ° C. for 10 hours. Moreover, a vaccination dosage is based on known dosages for poxvirus, such as vaccinia virus (e.g., prophylaxis vaccination for small pox).

In view of the above, the advantages of the embodiments herein over current technology are increased safety of live vaccine vectors and increased immunogenicity.

The claims are not intended to be limited to the embodiments and examples described herein.

What is claimed is:

1. A vaccine composition including a heat-inactivated poxvirus.

2. The vaccine composition of claim 1, wherein said vaccine composition induces interferon in a recipient upon administration of said vaccine composition.

3. The vaccine composition of claim 1, wherein said heat-inactivated poxvirus comprises a vaccinia virus.

4. The vaccine composition of claim 2, wherein said heat-inactivated poxvirus comprises a vaccinia virus.

5. The vaccine composition of claim 1, further comprising a mixture of said heat-inactivated poxvirus and one or more of a live vaccine vector and a non-replicating vaccine component.

6. The vaccine composition of claim 5, wherein said heat-inactivated poxvirus comprises a vaccinia virus.

7. A method for preparation of a vaccine, comprising the step of adding a heat-inactivated poxvirus to said vaccine prior to administration to a vaccine recipient.

8. The method of claim 7, wherein said heat-inactivated poxvirus comprises vaccinia virus.

9. The method of claim 7, further comprising adding one or more of a live vaccine vector and a non-replicating vaccine component to said vaccine.

10. A vaccination method, comprising administering a vaccine composition including a heat-inactivated poxvirus to an animal or human.

11. The vaccination method of claim 10, wherein said heat-inactivated poxvirus comprises a vaccinia virus.

12. The vaccination method of claim 10, further comprising a mixture of said heat-inactivated poxvirus and one or more of a live vaccine vector and a non-replicating vaccine component.
